(19) United States
(12) Patent Application Publication
(10) Pub. No.: US 2024/0025828 A1
(43) Pub. Date: Jan. 25, 2024

(54) PROCESS FOR PRODUCING ALCOHOLS

(71) Applicant: Neste Oyj, Espoo (FI)

(72) Inventors: Marja TIITTA, Porvoo (FI); Ulla KIISKI, Porvoo (FI); Anna KARVO, Porvoo (FI); Terhi KOLEHMAINEN, Porvoo (FI)

(73) Assignee: Neste Oyj, Espoo (FI)

(21) Appl. No.: 18/254,675

(22) PCT Filed: Nov. 16, 2021
(86) PCT No.: PCT/FI2021/050774

§ 371 Date: May 26, 2023

(30) Foreign Application Priority Data
Dec. 18, 2020 (FI) 20206328

(51) Int. Cl. C07C 29/16; C07C 29/82; B01D 3/36; B01D 3/14; C07C 4/04; C10G 65/12

(52) U.S. Cl. CPC C07C 29/16; C07C 29/82; B01D 3/36; B01D 3/14; C07C 4/04; C10G 65/12

(57) ABSTRACT

A process for converting steam cracking products into an alcohol composition consisting essentially of 2-propanol and butanols is disclosed. The process includes steam cracking and recovery of a stream including propene and butenes, which are next converted catalytically in the presence of water to 2-propanol and butanols respectively.

### TECHNICAL FIELD

The present disclosure generally relates to processes for cracking hydrocarbons and converting the products thereof to higher value products. The disclosure relates particularly to converting the cracking products to alcohols. Especially, although not exclusively, the disclosure relates to cracking feeds comprising renewable hydrocarbons, which hydrocarbons are derived from hydrotreatment of renewable fats and/or oils. The present disclosure is also related to oxygen containing gasoline components and further to gasoline blends, where at least one of the components therein contains oxygen.

### BACKGROUND

This section illustrates useful background information without admission of any technique described herein representative of the state of the art.

Gasoline is a mixture of volatile, flammable liquid hydrocarbons and used as fuel for internal combustion engines. Gasoline is a complex mixture of different hydrocarbons, which each contribute to the properties of the product.

Additives to gasoline often include detergents to reduce the buildup of engine deposits, and antioxidants (oxidation inhibitors) used to reduce “gum” formation. A well-known solution for gasoline is composed of 10 vol-% ethanol and 90 vol-% unleaded gasoline. Alternatives for future gasoline components providing biocontent and oxygenates are constantly studied.

Particulate emissions are of interest also in gasoline engines, especially in modern direct injection (GDI=Gasoline Direct Injection, DISI=Direct-Injection Spark Ignition) engines. The particulate emissions are controlled according to emission regulation Euro 6c category, in force since 2017. Particulate emissions can be either managed with engine technologies using gasoline particulate filters, or to some extent, with fuels. Oxygenates are seen as one potential solution for decreasing particulate emissions.

Isopropanol is one of the oxygenates of interest. However, current processes for isopropanol production suit better for specialty chemical production than for producing isopropanol for a fuel component.

Isopropanol may be manufactured by hydrolysis of propene. There are several different methods for said reaction. Commercially two processes for producing isopropanol from propene are classified as indirect and direct hydrolysis. They are basically rather similar, the product compositions from the different reactions are alike, and the separation processes applied afterwards are identical.

In indirect hydrolysis, sulphuric acid is used as a catalyst. It proceeds through two reaction steps. At the first step, propene reacts with sulphuric acid, and isopropyl hydrogen sulphate is mainly formed. At the second step, isopropyl hydrogen sulphate reacts with water. Isopropanol and sulphuric acid are formed. The formed sulphuric acid is circulated back to the reactor.

The advantage of indirect hydrolysis is that the process is robust for the impurities of propene. The main disadvantages are that the post treatment of sulphuric acid is expensive, sulphuric acid is hazardous and corrosive, and waste treatment is costly.

In direct hydrolysis, catalyst is a solid acidic catalyst. The propene activates at the acidic site of the solid catalyst. The protonated propene is reacting with water to isopropanol.

The advantages of direct hydrolysis are that there are no hazardous wastes from catalyst. However, as a disadvantage of direct hydrolysis is considered the limited yields of known methods. The known solid catalysts work at temperatures above 150° C. The propene hydrolysis is equilibrium limited reaction and high isopropanol yield is possible at temperatures below 70° C. The increase of pressure increases the equilibrium yield of isopropanol. At pressure above 5 MPa, high yield of isopropanol is possible. Typical solid acid catalysts are ion exchange resins and zeolites.

Both the direct and indirect processes are reported in the literature requiring high propene purity, such as 95%, of the feed.

A prior art document, WO2019180584 A1 is concerned with a three-step process, comprising: 1) cracking C4 hydrocarbons into butadiene, 2) hydrogenation on butadiene into 1-butene and 2-butene, 3) conversion of 1-butene and 2-butene to alcohols. Nevertheless, said process does not produce propene nor combination of propanol with butanols. Another prior art document concerning butanol production, US2014066667 A1 is also silent about propanol production.

There still remains a need for production processes for oxygenate components usable in fuels, especially in gasoline. There still is a further need to provide high quality alternatives for ethanol as a gasoline component. There is further need for processes converting renewable hydrocarbon feeds to higher value renewable components.

### SUMMARY

It is an object of the present disclosure to provide an improved process for alcohol production using propene obtained from steam cracking hydrocarbons. It is a further object to provide an alcohol composition useful as a gasoline component in gasoline blends. It is yet another object to provide improved properties for gasoline blends through use of an alcohol composition according to the present invention.

As the first aspect of the present invention, herein is provided a process for producing 2-propanol and butanols comprising
a) providing a hydrocarbon feed;
b) steam cracking said hydrocarbon feed and recovery of a stream comprising propene and butenes;
c) catalytic conversion of propene and butenes in the presence of water to 2-propanol and butanols respectively,
d) recovery of an alcohol composition comprising a mixture of 2-propanol and butanols.

Surprisingly the present inventors have found the process according to the present invention producing a composition readily usable as a gasoline component in gasoline blends.

The use of ethene, a predominant product from steam cracker, as a polymer precursor is very common. However, other components originating from steam cracking, such as different C4-alkenes, C1-C4 alkanes, dienes, and heavier hydrocarbons, have been more difficult to use. Now, the present inventors have surprisingly found that a part of the product of steam cracker can be used for the manufacturing of alcohols, especially for isopropanol, with the simultaneous purification of the product stream. The product stream needs to be purified from dienes and other impurity compounds.

Embodiments of said process provide advantages.

As the second aspect of the present invention, herein is provided a product according to claim 11, an alcohol composition consisting essentially of 2-propanol and butanols, wherein the amount of 2-propanol is from 68 to 75 wt-% and the sum amount of 2-propanol and butanols is 99-100 wt-% of the total composition weight.

The present inventors have found the alcohol composition very promising as a gasoline component in gasoline blends. It has been found to provide desired properties to said gasoline blends, such as decreased vapour pressure, high octane numbers (RON, MON) and/or lowered particulate matter emissions.

Consequently, as the third aspect of the present invention is provided a use of the alcohol composition according to the present invention with a base gasoline for providing improved properties for gasoline blends.

Different non-binding example aspects and embodiments have been illustrated in the foregoing. The embodiments in the foregoing are used merely to explain selected aspects or steps that may be utilized in different implementations. Some embodiments may be presented only with reference to certain example aspects. It should be appreciated that corresponding embodiments may apply to other example aspects as well.

### BRIEF DESCRIPTION OF THE FIGURES

Some example embodiments will be described with reference to the accompanying figures, in which:

FIG. 1 shows a schematic drawing of an example embodiment of the process of the present disclosure.

FIG. 2 shows a schematic drawing of catalytic conversion for alcohol production as a part of the overall process.

FIG. 3 shows a schematic drawing of an alternative approach for catalytic conversion for alcohol production as a part of the overall process.

FIG. 4 shows a schematic view of a purification process using an entrainer for 2-propanol.

### DETAILED DESCRIPTION

In the following description, like reference signs denote like elements or steps.

All test method standards referred to in this text are the latest versions available at the filing date.

The process of the present invention is first described by reference to the process steps:
a) providing a hydrocarbon feed;
b) steam cracking said hydrocarbon feed and recovery of a stream comprising propene and butenes;
c) catalytic conversion of propene and butenes in the presence of water to 2-propanol and butanols respectively,
d) recovery of an alcohol composition comprising a mixture of 2-propanol and butanols.

Hydrocarbon Feed

In the present process, the feed to the steam cracking is referred to as a hydrocarbon feed. Said hydrocarbon feed is selected from renewable propane feed, a renewable naphtha feed, a renewable middle distillate feed, a fossil propane feed, a fossil naphtha feed, a fossil middle distillate feed or a combination thereof.

According to an embodiment, said hydrocarbon feed comprises renewable hydrocarbons, that is, at least a part of the hydrocarbons in said feed are of biological origin. According to a preferred embodiment said hydrocarbon feed is completely renewable feed, when it consists of renewable hydrocarbons, such as renewable propane feed, a renewable naphtha feed, a renewable middle distillate feed or a combination thereof. Hence all of the hydrocarbons present in the renewable hydrocarbon feed are of biological origin.

As used herein a renewable hydrocarbon feed refers to a composition derived from a renewable source or renewable sources and comprising to a large extent paraffins (non-cyclic alkanes), both linear normal paraffins (n-paraffins) and branched isoparaffins (i-paraffins). Said isoparaffins may be monobranched i-paraffins, di-branched i-paraffins, tri-branched i-paraffins, i-paraffins comprising more than three branches, or a combination thereof. Preferably, the isoparaffins are methyl substituted isoparaffins, i.e. isoparaffins wherein the side chain or sidechains, i.e. the branch or branches, are methyl sidechains.

According to a preferred embodiment, the hydrocarbon feed comprises or consists of naphtha. Said naphtha may comprise fossil naphtha, renewable naphtha or a combination thereof. If defined by boiling points, the hydrocarbons of said naphtha feed boil mainly between about 40° C. and about 170° C.

As to hydrocarbon structure, the naphtha feed consists substantially of hydrocarbons having a carbon number from C4 to C9. The use of hydrocarbons having a carbon number from C4 to C9 as the hydrocarbon feed provides advantages through compatibility and association to adjacent processes within a refinery.

Preferably the hydrocarbon feed consists of renewable naphtha. With renewable naphtha is herein referred to a hydrocarbon stream consisting of hydrocarbons obtainable from renewable sources.

As used herein, the term “renewable naphtha feed” is intended to mean a mixture of C4 to C9 hydrocarbons (CₙH₂ₙ₊₂, n=4, 5, 6, 7, 8 or 9), i.e. straight or branched hydrocarbons having 4 to 9 carbon atoms originating from renewable sources such as e.g. plant/vegetable oil or animal fat and consequently not derived from any fossil based material. Such hydrocarbons may be n-alkanes and/or iso-alkanes. Consequently, the renewable naphtha component may comprise a mixture of one or more of n-hexane, n-pentane, 2-methylbutane (iso-pentane) and other C4 to C9 alkanes such as e.g. 2-methyl pentane, 2,3-dimethyl butane, heptane, 3-methyl hexane.

Alternatively or additionally, the hydrocarbon feed may be selected from a propane feed or a middle distillate feed, when it consists substantially of hydrocarbons having a carbon number of C3 or from C8 to C24 respectively, of fossil or renewable origin or a combination thereof.

According to an embodiment, the hydrocarbon feed comprises middle distillate, which here refers to a hydrocarbon stream consisting of hydrocarbons in the middle distillate range obtainable from fossil or renewable sources, or a combination thereof. If defined by boiling points, the hydrocarbons of said middle distillate feed boil mainly between about 170° C. and about 360° C.

When the hydrocarbon feed comprises renewable hydrocarbons, the renewable hydrocarbon feed may be obtained by:
(i) providing a renewable fresh feed of renewable oil(s) and/or fat(s);
(ii) subjecting a hydrotreatment feed comprising the renewable fresh feed and an optional diluent to hydrotreatment comprising at least hydrodeoxygenation to provide a hydrotreatment effluent; and
(iii) subjecting the hydrotreatment effluent to gas-liquid separation to provide a gaseous hydrotreated fraction and a liquid hydrotreated fraction comprising paraffins;
(iii) subjecting the liquid hydrotreated fraction comprising paraffins to fractional distillation wherefrom a stream, such as renewable propane feed, a renewable naphtha feed, a renewable middle distillate feed usable as the renewable hydrocarbon feed is recovered.

Hydrotreatment refers to reactions in the presence of hydrogen such as hydrodeoxygenation (HDO), hydrogenation of double bonds, hydrocracking and/or hydroisomerisation, and it may also remove some metals. Within the context of the present process, hydrotreatment is needed for olefinic bond saturation and for removal of covalently bound oxygen from the fatty acid ester molecules. Typically, this means deoxygenation by hydrogenation i.e. hydrodeoxygenation (HDO). Preferably, hydrotreatment comprises both hydrodeoxygenation and hydroisomerisation.

In one embodiment the hydrodeoxygenation takes place at reaction conditions comprising a temperature in the range from 100 to 500° C., preferably from 250 to 400° C., more preferably from 280-350° C., most preferably at temperature of 300-330° C.; and at a pressure in the range from 0.1 to 20 MPa, preferably from 0.2 to 8 MPa. Preferably, the weight hourly space velocity (WHSV) is in the range from 0.5 to 3.0 h-1, more preferably from 1.0 to 2.5 h-1, most preferably from 1.0 to 2.0 h-1. Preferably, H₂ flow is in the range from 350 to 900 nl H₂/l feed, more preferably from 350 to 750, most preferably from 350 to 500, wherein nl H₂/l means normal liters of hydrogen per liter of the feed into the HDO reactor, in the presence of a hydrodeoxygenation catalyst. The hydrodeoxygenation catalyst is preferably selected from Pd, Pt, Ni, Co, Mo, Ru, Rh, W, or any combination of these, such as CoMo, NiMo, NiW, CoNiMo on a support, wherein the support is preferably alumina and/or silica, preferably CoMo or NiMo on alumina support.

In one embodiment, the hydrotreatment is hydrodeoxygenation (HDO), or catalytic hydrodeoxygenation (catalytic HDO). The hydrotreatment is preferably performed at a pressure selected from the range 1-15, preferably 2-12 MPa, more preferably 3-10 MPa, and at a temperature selected from the range 200-400° C., preferably 250-380° C., more preferably 280-360° C. The hydrotreatment may be performed in the presence of known hydrotreatment catalysts containing metals from Group VIII and/or VIB of the Periodic System. Preferably, the hydrogenation catalysts are supported Pd, Pt, Ni, NiMo or a CoMo catalyst, the support being alumina and/or silica. Examples of typical catalysts for hydrodeoxygenation are molybdenum containing catalysts, such as NiMo, CoMo, CoNiMo, or NiW catalysts, supported on alumina or silica. The hydrodeoxygenation is preferably carried out under the influence of sulphided NiMo or sulphided CoMo or NiW catalysts in the presence of hydrogen gas. Typically, NiMo/Al₂O₃ and CoMo/Al₂O₃ catalysts are used.

In the hydroisomerisation, the temperature varies between 200-500° C., such as 280-400° C., such as 280-370° C., such as 300-370° C., such as 340-370° C. In a specific embodiment, the hydroisomerisation is performed at a temperature of 300° C. or above, preferably at 300-370° C., such as 340-370° C. The reaction conditions may further comprise a pressure in the range from 2 and 15 MPa, preferably between 2 and 10 MPa; a WHSV in the range from 0.5 to 3 h⁻¹, a H₂ flow in the range from 100 to 800 nl H₂/l feed, or a combination thereof.

Both the hydrodeoxygenation and hydroisomerisation may be conducted in the same reactor, and even in the same reactor bed. The hydroisomerisation catalyst may be a noble metal bifunctional catalyst such as a Pt containing commercial catalyst, for example Pt-SAPO or Pt-ZSM-catalyst or for example a non-noble catalyst, such as NiW. The hydrodeoxygenation and hydroisomerisation may be performed using NiW catalyst, or even in the same catalyst bed using the NiW catalyst for both the hydrodeoxygenation and isomerisation. The NiW catalyst may additionally result in more hydrocracking to renewable middle distillate and naphtha streams, both usable as the hydrocarbon feed of the present process.

The stream suitable as the hydrocarbon feed, more specifically a renewable hydrocarbon feed of the present process may be obtained as depicted e.g. in EP1741768A1, WO2007068795A1, WO2016062868A1 or EP2155838B1, and using a conventional hydrotreatment catalysts and hydrogen gas. In case the hydrotreatment comprises both hydrodeoxygenation and hydroisomerisation, said hydroisomerisation can be carried out in a conventional hydroisomerisation unit, such as those depicted in F1100248B, EP1741768A1, WO2007068795A1, WO2016062868A1 or EP2155838B1. Hydrogen is added into the isomerisation step.

The fresh feed of renewable oil(s) and/or fat(s), thus the oils and fats of biological origin and waste materials, and processing conditions in hydroprocessing effect the hydrocarbon distribution of the product obtainable thereof, which may be fractionated to obtain the desired hydrocarbon feed. The process optimisation is discussed for example in EP1741768A1.

Steam Cracking

Steam cracking is an important method, traditionally known for producing chemicals from fossil hydrocarbons. Examples of valuable products of a high severity fossil naphtha cracker are ethene, propene, 1,3-butadiene and BTX (benzene, toluene, xylenes). Steam cracking is the main source of raw materials for conventional petrochemistry, and in particular for the polymer industry. Major polymers such as polyethene (PE), polypropene (PP), and polyethylene terephthalate (PET) are conventionally obtained from raw materials produced by steam cracking fossil hydrocarbons. Recently, it has been suggested to replace at least a portion of the fossil raw materials conventionally used as steam cracker feedstock with more sustainable raw materials derived from renewable sources to address environmental concerns.

The processing conditions of steam cracking are well known, the implementation of the present invention thus requiring only few modifications of established processes. Thermally cracking the hydrocarbon feed is preferably performed in a conventional naphtha (steam) cracker, i.e. a cracker commonly used for thermally cracking fossil naphtha. The steam cracking is preferably carried out without catalyst. However, additives, particularly sulfur additives, may be used in the steam cracking step. The process of the present invention may comprise providing a steam cracking feedstock comprising sulfur to reduce coke formation, and to further reduce the formation of CO and CO₂ in the steam cracking step.

The steam cracking may be performed at a ratio of water to the steam cracking feedstock (H₂O flow rate [kg/h]/steam cracking feedstock flow rate [kg/h]) of 0.05-1.20, preferably 0.10-1.00, further preferably 0.20-0.80, more preferably 0.25-0.70, even more preferably 0.25-0.60, and most preferably 0.30-0.50. In certain embodiments, the steam cracking may be performed at a ratio of water to the steam cracking feedstock (H₂O flow rate [kg/h]/steam cracking feedstock flow rate [kg/h]) of 0.30-0.50 and at a coil outlet temperature (COT) selected from the range from 780° C. to 830° C. The coil outlet pressure in the steam cracking step may be selected from the range 0.09-0.3 MPa, preferably at least 0.1 MPa, more preferable at least 0.11 MPa or 0.12 MPa, and preferably at most 0.25 MPa, more preferably at most 0.22 MPa or 0.20 MPa.

The steam cracking process may comprise recycling unconverted reactants back to the steam cracking furnace. Optionally, certain less valuable portions of the cracking product, such as propane and ethane, may be recycled back to the steam cracking furnace to be converted to more valuable products, such as ethene and propene. Recycling unconverted reactants, less valuable portions of the cracking product, or both, increases the overall profitability and the overall yield of the steam cracking process.

The steam cracking produces a wide range of hydrocarbon products as to carbon numbers and functionalities. For the present disclosure, for specific interest are alkenes heavier than ethene, namely propenes and butenes. The content of methane, ethene, propene, butenes and so on in the steam cracker output, may be determined in accordance with ASTM D 2163.

Reaction Steps Following Steam Cracking

The steam cracking reaction is followed by quenching and cooling the cracking product. Typically, CO, CO₂, C₂H₂, or a combination thereof, is removed from the cracking product during the quenching and cooling. The process further comprises fractionating the remaining cracking product.

The cracking product is directed to demethanization, for methane recovery and conducting other hydrocarbons, C2 and C2+ further. The next step, deethanization of the demethanized steam cracking product, removes ethane and ethene, which are directed to other processes.

Hence in the overall process, step b) further comprises demethanization and deethanization of the steam cracking product prior to recovery of the stream comprising propene and butenes obtained as the top cut of the depropanizer, also referred to as C3+ cut. The bottom cut from the depropanizer comprising heavier hydrocarbons, referred to as C5+ (including C5, C6 and so on), may be recycled back to steam cracking.

Within the context of the cracking process, the C3+ cut is used to differentiate between the C2 cut and hydrocarbons of longer carbon chains of the cracking product stream. As mentioned above, said C2 cut, essentially consisting of ethene, is a relevant product as such but is not related to the present process.

A typical composition of the top cut of the depropanizer, hence the stream comprising propene and butenes may be defined comprising 65-75 wt-% propene, 0.5-2 wt-% propane, 25-30 wt-% butenes and 0.1-1 wt-% butane by the total weight of the top cut of the depropanizer.

The bottom cut of deethanizer is conducted to depropanizer, from which the top cut provides propene and butenes in a specific proportion which has been shown advantageous when converted further to alcohols. Under the process conditions, a cracking product may comprise traces of unconverted starting materials, as well as some side products, such as dienes. Preferably the amount of said unconverted starting materials and side products is low in the bottom cut of deethanizer.

Conversion to a Mixture of 2-Propanol and Butanols

The present inventors have found that C3-hydrogenation step of the conventional process can be disregarded and C3 and C4 alcohols can be manufactured from the top cut of depropanizer, when propene and butenes are converted to respective alcohols 2-propanol and butanols.

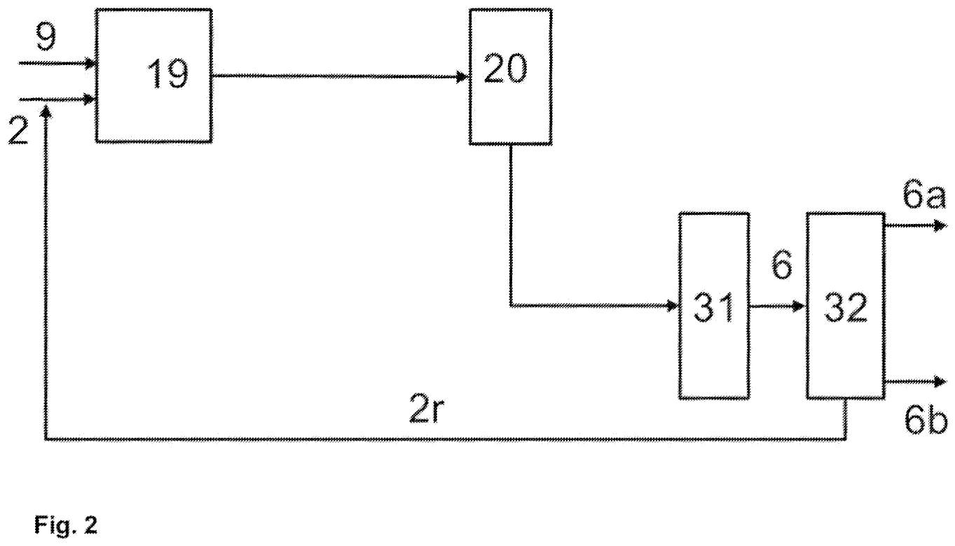

The steam cracking product, hence the stream comprising propene and butenes is catalytically converted in the presence of water to 2-propanol and butanols respectively. Preferably the catalytic conversion (also referred as hydration reaction) of propene and butenes is conducted in the presence of water in the liquid phase with ratio from 10:1 to 14:1, preferably about 12:1, water to alkene feed. The process scheme for catalytic conversion is shown in FIG. 2.

This feed is introduced to the reactor packed with a water tolerant solid acid catalyst. Water is added to the reaction as a reactant and solvent. Water reacts with propene and butenes as well as propyne, propadiene and butadienes. Propanols and butanols are formed. Based on Markonikow's rule, the main product form propene is 2-propanol (isopropanol). Butenes produce butanols.

As used herein, butanols refer to C4-alcohols, namely 1-butanol, 2-butanol and i-butanols. In the catalytic conversion 1-butene produces 1-butanol and 2-butanol, i-butene produces i-butanol and cis- and trans-2-butenes produce 2-butanol. 2-butanol is the most desirable component for gasoline compositions. However, since the i-butanol is not preferred as a gasoline component, it may be separated from the alcohol composition of the present disclosure, or alternatively, i-butanols may be removed from the propenes and butenes before catalytic conversion into alcohols. In such embodiments, butanols are practically free from i-butanol and consist essentially of 1-butanol and 2-butanol.

The catalytic conversion of propene and butenes may be conducted at reaction conditions comprising a temperature from 70° C. to 100° C., pressure from 3 to 7 MPa, or a combination thereof. The preferable conditions are reaction temperatures below 90° C. and pressure below 5 MPa to have high yield. If the yield of propanol is not sufficient, catalytic distillation unit can be considered where the unreacted propene is circulated back to the reaction zone.

Propene and butenes are converted to respective alcohols 2-propanol and butanols in the presence of a catalyst. A mixture of 2-propanol and butanols, preferably in proportion form 2.3:1 to 3:1 is recovered.

The feedstock of reactor system producing alcohol can have C3+ cut as a feedstock. As the feedstock contains both propene and butene, the product stream of process will contain both propanols and butanols. In this type of process, the catalytic distillation reactor would be very beneficial to provide the control of concentrations in every catalyst zone and simultaneously separation of formed alcohols to overcome equilibrium conversion in total process.

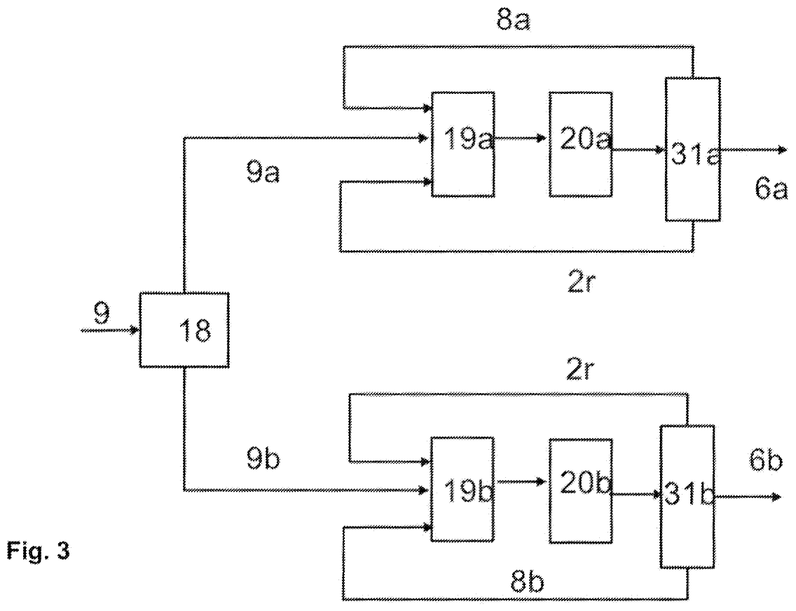

When 2-propanol and butanols are needed to be produced separately, C3 and C4 streams can be separated before the hydration reactions. Such process scheme is shown in FIG. 3. The 2-propanol and butanols may be combined afterwards to provide an alcohol composition in desired proportions.

Water is recycled in its own stream, but it is not possible to separate all of it from the stream containing the products as it forms azeotropes with the products. Nevertheless, a high amount of water can still be recycled which will lower the amount of water fed into the process substantially.

Recovery of a Mixture of 2-Propanol and Butanols

When separating the olefin reactants from the reactor outflow through distillation, it was discovered that the ethers (such as di-isopropyl ether, di-secondary butyl ether and isopropyl secondary butyl ether) were also separated quite easily in the distillate side of the column, and it is not possible to separate propene and other components entirely without having some amount of ethers in the distillate stream as well. Hence, olefin reactants and said ethers are separated in the same column and recycled back into the catalytic conversion reactor. This also further reduces the number of process equipment in the process as there is no need for a separate column for removing ethers.

After removal of olefins and ethers, the product stream still contains a significant water content. Alcohols, such as 2-propanol and butanols are known to form azeotropes with water. However, there are many different alternatives studied and utilized, such as azeotropic distillation with different entrainers, salting out and pressure swing distillation for 2-propanol and butanols purification. Preferably purification 2-propanol and butanols is conducted by distillation with entrainers.

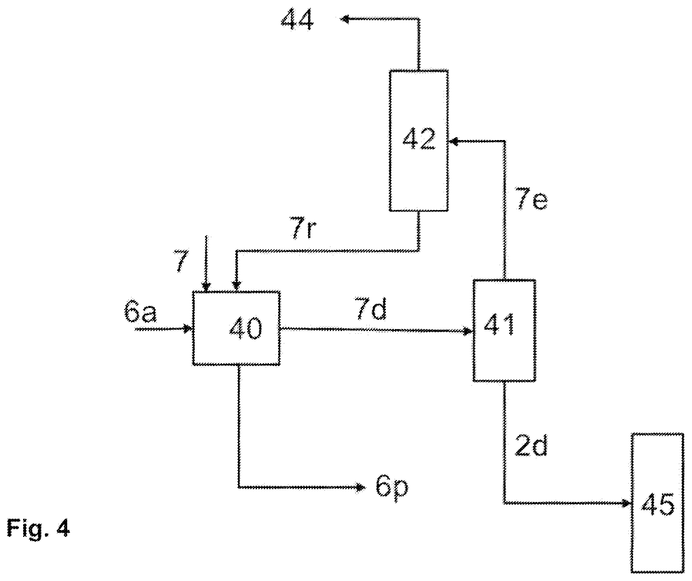

FIG. 4 shows the schematic view of the purification process using entrainers, depicting 2-propanol as an example.

Proportions

The present inventors have surprisingly found that the process converting naphtha through steam cracking followed by a catalytic conversion provides a novel composition of 2-propanol and butanols, wherein said two alcohols may be recovered in a ratio of 2-propanol to butanols of about 2.5:1, such as from 2:1 to 3.5:1, or from 2:1 to 3:1. The present process directly yields the alcohol composition meeting said ratio.

Product

According to the second aspect of the present invention, a novel alcohol composition consisting essentially of 2-propanol and butanols is provided, wherein the amount of 2-propanol is from 68 to 75 wt-% and the sum amount of 2-propanol and butanols is 99-100 wt-% of the total composition weight.

The present alcohol composition may be used as a gasoline component in a blend with a base gasoline. Such a blend comprising the alcohol composition consisting essentially of 2-propanol and butanols meets requirements of gasoline standards, such as EN228 (2017).

The base gasoline without oxygen content may be a combination of hydrocarbons comprising paraffins, aromatics and olefins having about 4 carbon atoms or more, such as 4 to 12 carbon atoms. The base gasoline without oxygen may have a boiling point in the range from about 30° C. to about 230° C., or preferably from about 30° C. to about 210° C.

In one aspect, the base gasoline without oxygen content may be present in a gasoline blend in an amount in the range from e.g. about 50 vol % to about 95 vol %, such as e.g. about 65 vol % to about 95 vol %, such as e.g. about 70 vol % to about 95 vol %, such as e.g. about 75 vol % to about 95 vol %, such as e.g. about 80 vol % to about 95 vol %, such as e.g. about 85 vol % to about 95 vol %, such as e.g. about 88 vol % to about 95 vol %, or such as e.g. about 60 vol % to about 95 vol % of the total gasoline blend weight.

The alcohol composition may be present in a gasoline blend as an amount of e.g. about 5 vol % to about 15 vol % based on the total gasoline blend, such as e.g. about 10 vol % to about 15 vol % based, such as e.g. about 5 vol %, about 10 vol %, about 12 vol %, or about 15 vol % of the total gasoline blend volume.

According to a specific embodiment, the gasoline blend has an oxygen content of 3.7 wt-% of the total gasoline blend weight.

As used herein, the “total” gasoline blend means the finished composition, wherein all components mentioned herein, and in the attached claims have been mixed together. Usually, the terminology entails the vol % (volume percentage; VN) but may also be wt-% (weight percentage; m/m) as appropriate and as indicated in each instance.

The present alcohol composition when used as a component in a gasoline blend has been found to contribute positively to at least one of the gasoline blend properties selected from vapour pressure, RON, MON, particulate matter emissions. More specifically, the present inventors found the present alcohol composition reducing vapour pressure.

The ability of present alcohol composition to reduce particulate matter emissions may be estimated by particulate matter index. The particulate matter index is a recognized equation suitable for evaluating particulate emissions of different fuels or components. Particulate matter index uses gasoline composition parameters such as vapour pressure and component fractions as given in formula 1 below.

$$PMI = {\sum }_{i = 1}^{n} ( \frac{{DBE}_{i} + 1}{V . {P ( 443 K )}_{i}} * {Wt}_{i} ) \qquad ( 1 )$$

wherein
DBE=double bond equivalent which can be calculated based on the number of the different types of atoms present in the molecule (2C+2-H+N)/2
V.P(443 K)=the vapour pressure of the component at 443 K (170° C.)
Wt=the fractional weight contribution of the component to the fuel.

Using this calculation, the present inventors found the alcohol composition consisting essentially of 2-propanol and butanols, wherein the amount of 2-propanol is from 68 to 75 wt-% and the sum amount of 2-propanol and butanols is 99-100 wt-% of the total composition weight providing reductions to particulate matter emissions.

Further, the present inventors found the present alcohol composition increasing research octane number (RON) as well as motor octane number (MON).

The anti-knock quality of a fuel is normally rated by its octane number (ON), which can be determined in accordance with one of two protocols on a so-called cooperative fuel research (CFR) engine: Research Octane Number (RON) or Motor Octane Number (MON). Both standards use n-heptane and iso-octane as reference fuels. To date, both values are determined on a standardized CFR engine in accordance with ASTM protocols D-2699 and D-2700, respectively. Both norms were designed to be representative of the mildest (RON) and most severe (MON) operating conditions. In both tests, the highly reactive n-heptane and highly stable iso-octane are used as surrogate fuels, spanning the octane scale from 0 to 100, respectively.

The modern gasoline specifications typically require RON to be at least 95 and MON 85. Literature provides typically different octane boosting agents to raise the octane numbers to desired level.

According to the invention, the gasoline blend may have a RON (research octane number) which may be e.g. at least about 95 or higher, such as e.g. at least about 98 or higher.

According to the invention, the gasoline blend may have a MON (motor octane number) which may be e.g. at least about 85 or higher.

Using the measured RON and MON for the blends and for each blend components, blending RON and MON ratings (denoted as bRON and bMON) can be calculated for components. Calculating bRON is known in the field and has been published for example in U.S. Pat. No. 4,244,704A.

The product characteristics are further discussed in relation to the experimental part.

### Terminology

The term hydrotreatment, sometimes also referred to as hydroprocessing, refers herein to a catalytic process of treating organic material by means of molecular hydrogen. Preferably, hydrotreatment removes oxygen from organic oxygen compounds as water i.e. hydrodeoxygenation (HDO), removes sulphur from organic sulphur compounds as dihydrogen sulphide (H₂S), i.e. hydrodesulphurisation, (HDS), removes nitrogen from organic nitrogen compounds as ammonia (NH₃), i.e. hydrodenitrogenation (HDN), removes halogens, for example chlorine from organic chloride compounds as hydrochloric acid (HCl), i.e. hydrodechlorination (HDCI), removes metals by hydrodemetallization, and hydrogenates any unsaturated bonds present in the fresh feed. As used in the context of this disclosure, hydrotreatment covers or encompasses also hydroisomerisation.

The term hydrodeoxygenation (HDO) refers in the context of this disclosure to removal of oxygen from organic molecules as water by means of molecular hydrogen under the influence of catalyst.

The term deoxygenation refers in the context of this disclosure to removal of oxygen from organic molecules, such as fatty acid derivatives, alcohols, ketones, aldehydes or ethers by any means previously described or by decarboxylation or decarbonylation.

The term renewable or bio-based or biogenic indicates presence of compounds or components derived from renewable sources (biological sources). Carbon atoms of renewable or biological origin (biogenic carbon) comprise a higher number of unstable radiocarbon (¹⁴C) atoms compared to carbon atoms of fossil origin.

Therefore, it is possible to distinguish between carbon compounds derived from renewable or biological raw material and carbon compounds derived from fossil raw material by analysing the ratio of ¹²C and ¹⁴C isotopes. Thus, a particular ratio of said isotopes can be used as a “tag” to identify renewable carbon compounds and differentiate them from non-renewable carbon compounds. The isotope ratio does not change in the course of chemical reactions. Examples of a suitable method for analysing the content of carbon from biological or renewable sources are DIN 51637 (2014), ASTM D6866 (2020) and EN 16640 (2017). In the context of the present disclosure, the content of carbon from biological or renewable raw material (biological origin) is expressed as the biogenic carbon content meaning the amount of biogenic carbon in the material as a weight percent of the total carbon in the material as determined according to EN 16640 (2017). A ¹⁴C isotope content of the total carbon content in a product, which is completely of biological origin, is about 100 wt-%. The ¹⁴C isotope contents of the hydrocarbon feed, intermediates, alcohols, specifically 2-propanol and butanols, according to the present disclosure are lower in cases where other carbonaceous components besides biological or renewable components are used in the processing of the product, but the ¹⁴C isotope contents are preferably at least 5 wt-%.

The recovered alcohol composition may be a fully renewable alcohol composition or may be an alcohol composition which is a blend of renewable and fossil material. In certain embodiments, the biogenic carbon content of the alcohol composition is at least 5 wt-%, preferably at least 10 wt-%, at least 50 wt-%, at least 75 wt-%, at least 90 wt-% or at least 100 wt-% based on the total weight of carbon in the alcohol composition. In such embodiments, the feed is a fully renewable hydrocarbon feed (biogenic carbon content 100 wt-%).

Process Description with the Aid of the Figures

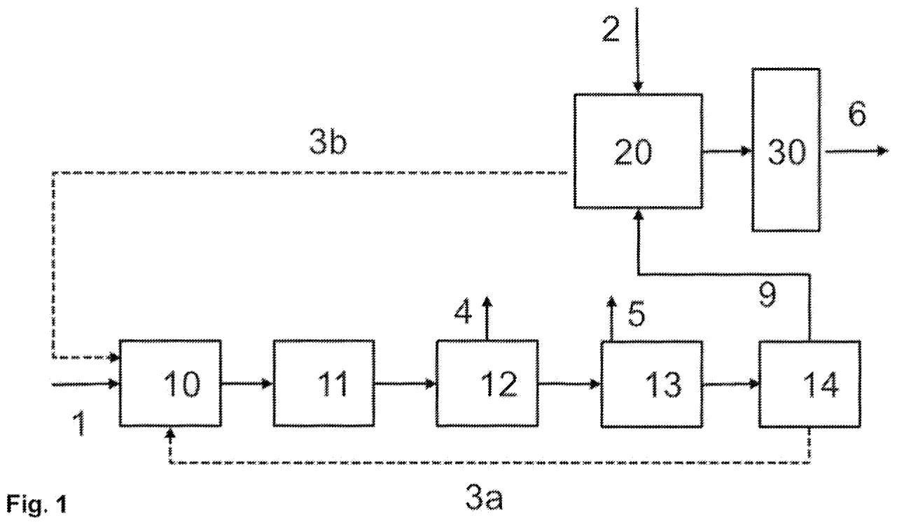

FIG. 1 shows a schematic drawing of the present process for producing 2-propanol and butanols. A hydrocarbon feed 1 is fed to the steam cracking reactor 10. The steam cracking product is fed via a caustic oxidation 11 to demethanizer 12. Methane 4 is recovered and the rest of the stream fed to deethanizer 13. After recovery of ethene together with some ethane, the bottom cut of the deethanizer 13, comprising propene and butenes, is fed to depropanizer 14. The top fraction 9 of depropanizer comprising propene and butenes, is next converted into alcohols via catalytic conversion in reactor 20. The bottom fraction of depropanizer is fed back to the steam cracking reactor 10 by combining this stream with stream 1. Water 2 is fed to reactor 20 in excess. Any propanes or butanes present in said alcohol conversion are recycled back to cracking 10 via recycle line 3b. Alternatively or additionally the C5+ fraction may be recycled back to cracking 10 via recycle line 3a from depropanizer 14 before entering the alcohol conversion 20. From separation and recovery 30, product 6, an alcohol composition comprising 2-propanol and butanols is recovered.

FIG. 2 shows a schematic simplified drawing of another process for producing 2-propanol and butanols. A hydrocarbon feed is steam cracked, the product thereof treated in a caustic oxidation, demethanizer, deethanizer and depropanizer (not shown). A stream comprising propene and butenes 9 is mixed with excess water 2 in mixer 19. The conversion into alcohols takes place in the catalytic conversion in reactor 20. The separation and recovery are arranged so that water is first separated from the catalytic conversion product in water distillation 31, wherefrom water is recycled via line 2r back to the mixer 19. As water is removed, product 6, an alcohol composition comprising 2-propanol and butanols is recovered. In FIG. 2, azeotropic distillation 32 provides an alternative process option for separating 2-propanol 6a from butanols 6b.

FIG. 3 provides yet another schematic drawing of a further process for producing 2-propanol and butanols. Again, a hydrocarbon feed is obtained by steam cracking, followed by appropriate treatments to obtain a stream comprising propene and butenes (not shown). A stream comprising propene and butenes 9 is first separated into propene 9a and butenes 9b. Each alcohol is then mixed with water in mixers 19a for propene and 19 b for butenes, from which fed to catalytic conversion reactors 20a and 20b respectively. The separation and recovery are again arranged to remove water first in water distillation 31a and 31b, wherefrom water is recycled via line 2r back to the mixers 19a and 19b. FIG. 3 provides for recycle via recycle lines 8a and 8b of unreacted propene and butenes respectively. Distillations in water distillators 31a and 31b provide as products 2-propanol 6a and butanols 6b respectively.

In FIG. 4, purification of 2-propanol azeotrope as an example of end product purification with an entrainer is schematically described. It can be considered as continuation to the process of FIG. 3, wherein water distillation column 31a and 31b (not shown in FIG. 4) provided 2-propanol 6a and butanols 6b as products respectively. As impurities the 2-propanol stream 6a may contain for example water, propene, propane and diisopropyl ether, DIPE. Since DIPE is one of the impurities formed in the catalytic conversion reaction, it's use as an entrainer is especially beneficial. In FIG. 4, stream 6a is fed to purification process together with fresh DIPE 7 as entrainer, starting with the first distillation column 40. The pure 2-propanol 6p is recovered from the bottom of the first distillation column 40, whereas impurities together with the entrainer are removed from the top of the column as distillate 7d. Next the decanter 41 separates two liquid phases from one another, namely water 2d from organic phase 7e. Water 7e is led to purification 45, whereform some 2-propanol may still be recovered (not shown), giving water pure enough to be used as recycle water or to be discarded into waste stream. The organic phase 7e comprises propane, propene and DIPE. The organic phase 7e separates into gases, which are purged via 44, and into pure DIPE, which is returned as recycle DIPE 7r back to purification distillation.

### EXAMPLES

The following examples are provided to better illustrate the claimed invention and are not to be interpreted as limiting the scope of the invention. To the extent that specific materials are mentioned, it is merely for purposes of illustration and is not intended to limit the invention.

Production of an alcohol composition consisting essentially of 2-propanol and butanols was studied. A renewable naphtha feed was steam cracked. The light components were separated, first methane in a demethanizer followed by separation of ethane and ethene in a deethanizer. The bottom cut from the deethanizer was fed to depropanizer, from which the bottom product was recycled back to steam cracker and the lighter stream, also referred to as the C3+ cut comprising propene and butenes, was next subjected to catalytic conversion in the presence of water. Propene and butenes yielded 2-propanol and butanols respectively. The recovery of a mixture of 2-propanol and butanols exhibits a characteristic ratio therein. Said ratio is somewhat dependent on the conversion

In the present examples, the biogenic carbon content of alcohol and consequently of the alcohol composition provided as the end product were about 100 wt-% based on the total weight of carbon (TC) in the renewable hydrocarbon feed and the alcohol composition respectively as determined according to EN 16640 (2017).

### Example 1 Production of an Alcohol Composition According to the Present Invention

Steam cracking experiments illustrating certain embodiments of the present invention where carried out on a bench scale equipment. The main parts of the steam cracking unit, the analytical equipment and the calibration procedure used in these examples have been described in detail in the following publications K. M. Van Geem, S. P. Pyl, M. F. Reyniers, J. Vercammen, J. Beens, G. B. Marin, On-line analysis of complex hydrocarbon mixtures using comprehensive two-dimensional gas chromatography, Journal of Chromatography A. 1217 (2010) 6623-6633 and J. B. Beens, U. A. T. Comprehensive two-dimensional gas chromatography—a powerful and versatile technique. Analyst. 130 (2005) 123-127. Two different renewable isomeric paraffin compositions P1 and P2, as well as blends of said renewable isomeric paraffin compositions and fossil naphtha N1 were studied as steam cracking feedstocks. Further, as comparative examples, fossil naphtha N1, a third renewable isomeric paraffins composition P3, and a blend of the third renewable isomeric paraffin composition and fossil naphtha N1 were studied as steam cracking feedstocks.

The experiments were conducted using a bench scale equipment. The feed section controls the supply of the steam cracking feedstock and the water from to the reactor coil. The flow of liquids was regulated by coriolis flow meter controlled pumps (Bronkhorst, The Netherlands) equipped with Bronkhorst™ CORI-FLOW™ series mass flow metering instruments to provide high accuracy: ±0.2% of reading. CORI-FLOW™ mass flow metering instruments utilizes an advanced Coriolis type mass flow sensor to achieve reliable performance, even with changing operating conditions, e.g. pressure, temperature, density, conductivity and viscosity. The pumping frequency was automatically adjusted by the controller of the CORI-FLOW™ flow metering instrument. The mass flow rate, which contrary to the volume flow rate is not affected by changes in temperature or pressure, of all feeds was measured every second, i.e. substantially continuously. Steam was used as a diluent and was heated to the same temperature as the evapourated feedstock. Both the feedstock and the steam were heated in two electrically heated ovens. Downstream from the ovens, the feedstock and the steam were mixed in a further electrically heated oven filled with quartz beads, which enabled an efficient and uniform mixing of feedstock and the diluent prior to entering the reactor coil. The mixture of feedstock and diluent steam entered the reactor coil placed vertically in a rectangular electrically heated furnace. Eight thermocouples T positioned along the axial reactor coordinate measured the process gas temperature at different positions. The rectangular furnace was divided into eight separate sections which could be controlled independently to set a specific temperature profile. The pressure in the reactor coil was controlled by a back-pressure regulator (not shown) positioned downstream from the outlet of the reactor coil. Two pressure transducers (not shown), placed at the inlet and outlet of the reactor, indicated the coil inlet (CIP) and the coil outlet pressure (COP), respectively. At the reactor outlet, nitrogen was injected to the reactor effluent as an internal standard for analytical measurements and to a certain extent contribute to the quenching of the reactor effluent. The reactor effluent was sampled online, i.e. during operation of the steam cracking setup, at a high temperature (350° C.). Namely, via a valve-based sampling system and uniformly heated transfer lines a gaseous sample of the reactor effluent was injected into a comprehensive two-dimensional gas chromatograph (GC×GC) 9 coupled to a Flame Ionization detector (FID) and a Mass Spectrometer (MS). A high temperature 6-port 2-way sampling valve of the valve-based sampling system was placed in an oven, where the temperature was kept above the dew point of the effluent sample. Further downstream the reactor effluent was cooled to approximately 80° C. Water and condensed heavier products (pyrolysis gasoline (PyGas) and pyrolysis fuel oil (PFO)) were removed by means of a knock-out vessel and a cyclone, while the remainder of the effluent stream was sent directly to a vent. Before reaching the vent, a fraction of the effluent was withdrawn for analysis on a Refinery Gas Analyzer (RGA). After removal of all remaining water using a water-cooled heat exchanger and dehydrator, this effluent fraction was injected automatically onto the so-called Refinery Gas Analyzer (RGA) using a built-in gas sampling valve system (80° C.). Results from said analyzer are compiled in tables 1 and 2 depicting the effect of reaction temperatures on the product distribution applying different water contents in the feed.

A hydrocarbon feed steam cracked in the present example comprised renewable naphtha.

TABLE 1
Effect of reaction temperatures on steam cracking products.
The steam cracking effluent analysis, renewable naphtha as feedstock,
75 g/h H₂O feed. The results are expressed in wt-% based
on the total weight of the effluent.

| | Coil outlet temperature (° C.) | | | | |
|---|---|---|---|---|---|
| | 780 | 800 | 820 | 840 | 860 |
| Hydrocarbons g/h | 150 | 150 | 150 | 150 | 150 |
| H₂O g/h | 75 | 75 | 75 | 75 | 75 |
| Dilution gH₂O/HC | 0.5 | 0.5 | 0.5 | 0.5 | 0.5 |
| P + E wt % | 45.45 | 49.11 | 51.01 | 50.56 | 49.89 |
| P/E g/g | 0.82 | 0.72 | 0.62 | 0.50 | 0.37 |
| P/B g/g | 2.09 | 2.40 | 2.93 | 3.61 | 5.35 |
| H₂ | 0.66 | 0.75 | 0.87 | 1.00 | 1.14 |
| C₂H₄ | 24.95 | 28.47 | 31.56 | 33.71 | 36.36 |
| C₂H₆ | 4.19 | 4.40 | 4.31 | 4.12 | 3.94 |
| C₂H₂ | 0.17 | 0.28 | 0.44 | 0.64 | 0.93 |
| CH₄ | 9.97 | 12.23 | 14.55 | 16.52 | 18.98 |
| C₃H₈ | 0.67 | 0.66 | 0.61 | 0.51 | 0.38 |
| C₃H₆ | 20.50 | 20.63 | 19.45 | 16.84 | 13.53 |
| 1,3-C₃H₄ | 0.13 | 0.30 | 0.36 | 0.47 | 0.42 |
| t2-C₄H₈ | 0.87 | 0.77 | 0.55 | 0.36 | 0.17 |
| 1-C₄H₈ | 3.64 | 3.01 | 2.10 | 1.32 | 0.46 |
| i-C₄H₈ | 4.17 | 3.83 | 3.19 | 2.44 | 1.48 |
| c2-C₄H₈ | 1.12 | 0.98 | 0.78 | 0.56 | 0.41 |
| MeAc | 0.21 | 1.27 | 1.12 | 1.16 | 0.72 |
| 1,3-C₄H₆ | 3.66 | 4.21 | 4.63 | 4.38 | 3.84 |
| C5+ hydrocarbons | 25.10 | 17.99 | 14.92 | 15.00 | 16.27 |
| (sum wt %) | | | | | |

In this table, several isomers of C₄H₈ are analysed. Of these, 1-butene is the most abundant cracking product, but since i-butene, cis-2-butene and trans-2-butene are also present, they are referred here as butenes.

TABLE 2
Effect of reaction temperatures on steam cracking products.
The steam cracking effluent analysis, renewable naphtha as feedstock,
and lower, 52 g/h H₂O feed. The results are expressed in
wt-% based on the total weight of the effluent.

| | Coil outlet temperature (° C.) | | | | |
|---|---|---|---|---|---|
| | 780 | 800 | 820 | 840 | 860 |
| Hydrocarbons g/hr | 150 | 150 | 150 | 150 | 150 |
| H₂O g/hr | 52 | 52 | 52 | 52 | 52 |
| Dilution gH₂O/HC | 0.35 | 0.35 | 0.35 | 0.35 | 0.35 |
| P + E wt % | 46.01 | 50.47 | 50.72 | 49.40 | 47.30 |
| P/E g/g | 0.84 | 0.72 | 0.58 | 0.47 | 0.37 |
| P/B g/g | 2.06 | 2.52 | 3.28 | 3.76 | 6.11 |
| H₂ | 0.71 | 0.82 | 0.96 | 1.03 | 1.13 |
| C₂H₄ | 25.02 | 29.38 | 32.11 | 33.67 | 34.58 |
| C₂H₆ | 4.89 | 5.35 | 5.16 | 4.65 | 4.13 |
| C₂H₂ | 0.17 | 0.30 | 0.38 | 0.55 | 0.86 |
| CH₄ | 10.67 | 13.29 | 15.48 | 17.02 | 19.13 |
| C₃H₈ | 0.74 | 0.74 | 0.64 | 0.57 | 0.36 |
| C₃H₆ | 20.98 | 21.09 | 18.62 | 15.72 | 12.72 |
| 1,3-C₃H₄ | 0.05 | 0.30 | 0.34 | 0.35 | 0.32 |
| t2-C₄H₈ | 0.86 | 0.75 | 0.49 | 0.26 | 0.11 |
| 1-C₄H₈ | 3.87 | 2.71 | 1.50 | 0.87 | 0.30 |
| i-C₄H₈ | 4.38 | 3.96 | 2.97 | 2.52 | 1.37 |
| c2-C₄H₈ | 1.10 | 0.95 | 0.72 | 0.53 | 0.30 |
| MeAc | 0.52 | 1.41 | 0.82 | 0.46 | 0.84 |
| 1,3-C₄H₆ | 2.84 | 3.77 | 4.19 | 4.05 | 3.47 |
| C₅₊ hydrocarbons | 22.53 | 14.89 | 14.47 | 14.62 | 18.98 |
| (sum wt %) | | | | | |

The main products of steam cracker unit are ethene and propene. The propene contents of the product of steam cracker are between 10-25 wt-% at reaction temperatures between 780-860° C. Both alkenes are useful as source material for polymers. Raising the reaction temperatures seems to shift the product distribution towards lighter products, for example the methane content is nearly 20 wt-% at the highest temperatures. Therefore, in the present cracking process, the temperature is preferably between 780-830° C. Said temperatures provide the desired propene/butene ratio for the present process.

The steam cracking reaction was followed by quenching and cooling the cracking product removing CO, CO₂, and C₂H₂. The lightest components of the cracking product were next recovered in demethanizer, deethanizer. The bottom cut from the depropanizer, which may be referred to as C5+ cut, was separated. From the depropanizer, the top cut was fed to the alcohol conversion.

A top cut of depropanizer comprised 70 wt-% propene, 1 wt-% propane, 28.5 wt-% butenes and 0.5 wt-% butanes by the total weight of the bottom cut of deethanizer.

The alcohol conversion was conducted in a continuous process in gas-liquid phase. C3-C4 stream was fed in the gas phase and water in the liquid phase with 12:1 feed ratio for water. Recycle for unreacted propene and butenes was arranged, where side products for the process, DIPE, DSBE and IPSBE are recovered as well. The heterogeneous catalyst used was Amberlyst DT. The reaction conditions were about 160° C. and 8 MPa.

Two runs were assessed. The first was conducted for feed rate of 7600 kg/h. The conversion of alkenes was about 77%, yielding a mixture of alcohols with rates 2-propanol 5700 kg/h and butanols 2450 kg/h. The corresponding rates with feed amount 6000 kg/h in equilibrium, hence conversion of 97% were 5700 kg/h and 1900 kg/h respectively. It can be calculated that said process produced alcohol compositions wherein about 70 wt-% was 2-propanol and 30 wt-% butanols and about 75 wt-% 2-propanol and 25 wt-% butanols respectively.

### Example 2 Purification of 2-Propanol as Example

Purification of 2-propanol azeotrope as an example of end product purification was assessed with DIPE as an entrainer. The process is schematically presented in FIG. 4. As impurities the 2-propanol stream contained water, propene, propane and DIPE. In addition to advantages relating to DIPE already earlier discussed, DIPE as an entrainer to the present process is especially attractive since it could be produced as a batch from the components of the present process without need to rely on outside sources.

To the first distillation column, it was fed about 8400 kg/h of DIPE per 5830 kg/h 2-propanol and 820 kg/h water. The 2-propanol in purity>90 wt-% was recovered from the bottom of the first distillation column. Impurities, the entrainer and water were removed from the top of the column. Two liquid phases, the DIPE-phase and water phase were separated in a decanter from one another, The organic phase comprise propane and propene in addition to DIPE. After separation of the gases, DIPE was returned back to purification distillation.

### Example 3 Properties of Alcohols of the Present Invention as Oxygen Containing Gasoline Component

Some characteristics of the alcohols of interest were assessed in respect to properties required/valued in gasoline blends. Table 3 provides comparison of the properties of C3 and C4 alcohols in comparison to those for ethanol, the predominant oxygenate used in gasolines presently. Other alcohol components that might be present in the final product contribute to the end product gasoline blends.

TABLE 3
Properties of pure C3 and C4 alcohols and ethanol.

| Property | Ethanol | Propanol | i-propanol | Butanol | i-butanol |
|---|---|---|---|---|---|
| Melting point (° C.)* | −114.14 | −124.39 | −87.41 | −88.60 | −88.44 |
| Boiling point (° C.)* | 78.24 | 97.04 | 82.21 | 117.6 | 99.4 |
| Density (g/cm³)* | 0.7893 | 0.8048 | 0.7855 | 0.8148 | 0.8063 |
| Vapour pressure (kPa)* | 7.87 | 2.76 | 6.02 | 0.86 | 2.32 |
| Heat of combustion (kJ/g)* | 29.67 | 33.64 | 33.38 | 36.10 | 35.90 |
| Oxygen content (wt-%)* | 34.7 | 26.6 | 26.6 | 21.6 | 21.6 |
| RON** | 109 | 104 | 113 | 98 | 107 |
| MON** | 90 | 89 | 97 | 85 | 93 |

*Source: CRC Handbook of Chemistry and Physics, 98th Edition
**Source: Co-Optima Fuel Engine Optimization database. [https://fuelsdb.nrel.gov/fmi/webd#FuelEngineCoOptimization]. Visited 27 Oct. 2017.

As can be seen, isopropanol provides especially attractive octane numbers.

Further butanol properties and usefulness as a gasoline component were determined using the values given in table 4.

TABLE 4
2-Butanol characterization.

| Parameter | Value | | Source |
|---|---|---|---|
| Melting point | −114.0° | C. | Scifinder |
| Boiling point | 99.5° | C. | Scifinder |
| Density | 0.8063 | g/cm3 | Scifinder |
| Vapour pressure | 1.70 | kPa | Pubchem |
| Oxygen content | 21.6 | wt-% | calculated |

| | | |
|---|---|---|
| RON | 107 | Christensen et al., Energy Fuels |
| MON | 93 | 2011, 25, 4723-4733 |

Based on the values given in table 4, 2-butanol appears as an interesting gasoline component having RON close to that for ethanol and MON even higher than for ethanol.

### Example 4 Properties of the Alcohol Composition According to the Present Invention in Gasoline Blends

To assess the effect of the alcohol composition according to the present invention on specific gasoline properties, several blends were prepared and properties determined. The results are given below in tables 5, 6, and 7.

TABLE 5
Octane number studies on different blends of 2-butanol
with a 4 component surrogate batch 1 were compared. The
4-component surrogate batch 1, referred to as A, consisted
of: 2,2,4-Trimethyl pentane, 45.8 mol %; toluene 33 mol
%; n-heptane 14.2 mol %; 1-hexene 5.0 mol %.

| BLEND | RON | MON |
|---|---|---|
| A | 90.3 | 84.7 |
| 10 vol-% 2-butanol (11.6 wt-% 2-butanol) and A | 93.1 | 86.5 |
| 20 vol-% 2-butanol (21.1 wt-% 2-butanol) and A | 96.1 | 88.2 |
| 30 vol-% 2-butanol (31.5 wt-% 2-butanol) and A | 98.2 | 89.5 |

Source: Co-Optima Fuel Engine Optimization database. [https://fuelsdb.nrel.gov/fmi/webd#FuelEngineCoOptimization]. Visited 27 Oct. 2017.

Surprisingly, the average bRONc was 118 and bMONc was 102 for 2-butanol. Hence, 2-butanol as a blend component appears as an octane enhancer, which may be employed in alcohol compositions comprising 2-propanol and butanols.

TABLE 6
Vapour pressure studies on different blends
with blend base C with 2-butanol.

| | RVP or | |
|---|---|---|
| BLEND | DVPE, kPa | PMI |
| C | 80.05 | 1.062 |
| 10 vol-% 2-butanol (10.8 wt-% 2-butanol) and C | 80.53 | 0.96 |
| 20 vol-% 2-butanol (21.7 wt-% 2-butanol) and C | 77.08 | 0.86 |
| 30 vol-% 2-butanol (32.0 wt-% 2-butanol) and C | 72.81 | 0.77 |

The results show 2-butanol lowering the vapour pressure in blends with both B and C. Further, very interesting trend with regard to particulate matter index was also observed further confirming the suitability of 2-butanol as a blend component to gasolines.

To study further the vapour pressure behaviour in blends, symmetrical 2-propanol and 2-butanol blends with yet another blend base, D, were prepared.

TABLE 7
Vapour pressure studies on different blends
of D with 2-butanol and D with 2-propanol.

| | RVP or | | RVP or |
|---|---|---|---|
| BLEND | DVPE, kPa | BLEND | DVPE, kPa |
| 10 vol-% 2-butanol and D | 37.30 | 10 vol-% 2-propanol and D | 39.30 |
| 20 vol-% 2-butanol and D | 34.10 | 20 vol-% 2-propanol and D | 37.40 |
| 30 vol-% 2-butanol and D | 31.30 | 30 vol-% 2-propanol and D | 35.60 |

These results confirm that 2-butanol performs better than 2-propanol for vapour pressure lowering in blends.

Considering the alcohol component obtainable as a direct product from the process according to the present invention, following calculated examples may be presented as to the octane numbers:
RON (75% 2-propanol, 25% 2-butanol): 0.75*113+0.25*107=111.5
MON (75% 2-propanol, 25% 2-butanol): 0.75*97+0.25*93=95.25
RON (70% 2-propanol, 30% 2-butanol): 0.70*113+0.30*107=111.2
MON (70% 2-propanol, 30% 2-butanol): 0.70*97+0.30*93=95.1.

Hence, an alcohol component comprising both 2-propanol and butanols as a blend component appears to provide surprisingly good properties in gasoline blends.

Various embodiments have been presented. It should be appreciated that in this document, words comprise, include, and contain are each used as open-ended expressions with no intended exclusivity.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1-16. (canceled)

17. A process for producing 2-propanol and butanols, the process comprising:
a) providing a hydrocarbon feed;
b) steam cracking said hydrocarbon feed and recovering a stream including propene and butenes;
c) catalytically converting propene and butenes in a presence of water to 2-propanol and butanols respectively; and
d) recovering an alcohol composition comprising a mixture of 2-propanol and butanols.

18. The process according to claim 17, wherein the hydrocarbon feed comprises:
a renewable hydrocarbon feed, which renewable hydrocarbon feed is selected from at least one or more of a renewable propane feed, a renewable naphtha feed, a renewable middle distillate feed and/or a combination thereof.

19. The process according to claim 17, wherein step b) comprises:
demethanization and deethanization of a steam cracking product prior to recovery of the stream including propene and butenes.

20. The process according to claim 17, comprising:
performing the steam cracking at a ratio of water to the steam cracking feedstock (H₂O flow rate [kg/h]/hydrocarbon feed flow rate [kg/h]) of 0.05-1.20, and/or 0.10-1.00, and/or 0.20-0.80, and/or 0.25-0.70, and/or 0.25-0.60, and/or 0.30-0.50.

21. The process according to claim 17, comprising:
performing the steam cracking at a coil outlet temperature (COT), from 780 to 860° C., and/or from 780° C. to 830° C.

22. The process according to claim 17, comprising:
performing the steam cracking at a coil outlet pressure selected from a range 0.09-0.3 MPa, and/or at least 0.1 MPa, and/or at least 0.11 MPa or 0.12 MPa, and/or at most 0.25 MPa, and/or at most 0.22 MPa or 0.20 MPa.

23. The process according to claim 17, comprising:
conducting the catalytic conversion of propene and butenes in a presence of water in a liquid phase with a ratio from 10:1 to 14:1, and/or about 12:1, water to alkene feed.

24. The process according to claim 17, comprising:
purification of 2-propanol and butanols by distillation with entrainers.

25. The process according to claim 17, wherein a biogenic carbon content of an alcohol composition is at least 50 wt-%, and/or at least 75 wt-%, and/or at least 90 wt-%, and/or at least 100 wt-% based on a total weight of carbon in the alcohol composition.

26. The process according to claim 18, wherein providing the renewable hydrocarbon feed comprises:
(i) providing a renewable fresh feed of renewable oil(s) and/or fat(s);
(ii) subjecting a hydrotreatment feed including the renewable fresh feed and an optional diluent to hydrotreatment including at least hydrodeoxygenation to provide a hydrotreatment effluent;
(iii) subjecting the hydrotreatment effluent to gas-liquid separation to provide a gaseous hydrotreated fraction and a liquid hydrotreated fraction including paraffins; and
(iii) subjecting the liquid hydrotreated fraction including paraffins to fractional distillation wherefrom a stream usable as the renewable hydrocarbon feed is recovered.

27. An alcohol composition consisting essentially of 2-propanol and butanols, wherein:
an amount of 2-propanol is from 68 to 75 wt-%; and
a sum amount of 2-propanol and butanols is 99-100 wt-% of a total composition weight of the alcohol composition.

28. The alcohol composition according to claim 27, wherein the sum amount of 2-propanol and butanols is 99.5-100 wt-% of the total composition weight.

29. The alcohol composition according to claim 27, wherein the sum amount of 2-propanol and butanols is 99.9-100 wt-% of the total composition weight.

30. The alcohol composition according to claim 27, in combination as a gasoline component with a base gasoline.

31. The alcohol composition in the combustion according to claim 30, wherein the alcohol composition consisting essentially of:
2-propanol and butanols, wherein the amount of 2-propanol is from 68 to 75 wt-%; and the sum amount of 2-propanol and butanols is 99-100 wt-% of the total composition weight, contributes positively to at least one or more of gasoline blend properties selected from vapour pressure, RON, MON, and/or particulate matter emissions.

32. The alcohol composition in the combustion according to claim 31, wherein said gasoline blend property is reduced vapour pressure, increased RON, increased MON and/or reduced particulate matter emissions.
